(19) United States
(12) Patent Application Publication
(10) Pub. No.: US 2008/0035148 A1
(43) Pub. Date: Feb. 14, 2008

(54) Device And Method Of Isolating Bias Flow Using Partition Position

(76) Inventors: Bradley P. Fuhrman, Buffalo, NY (US); Mark S. Dowhy, West Seneca, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 11/836,321

(22) Filed: Aug. 9, 2007

Related U.S. Application Data
(60) Provisional application No. 60/821,840, filed on Aug. 9, 2006.

(51) Int. Cl. A61M 16/08

(52) U.S. Cl. 128/205.15

(57) ABSTRACT

Isolation devices and methods of controlling a partition are disclosed. Devices according to the invention have a housing disposed about a movable partition. The housing has respirator and patient sides on respective first and second sides of the partition. The housing also has: (a) a respirator orifice on the respirator side, adaptable to be in pneumatic communication with a respirator; (b) a patient inspiration orifice on the patient side, adaptable to be in pneumatic communication with a patient; (c) a bias inflow orifice on the patient side, adaptable to be in pneumatic communication with a source of inspiratory gas; and, (d) an expiration return orifice on the patient side. The devices may further include a partition position sensor, a CO2 scrubber, and a controller operable to regulate gas flow to the bias inflow orifice based on partition position.

### CROSS REFERENCE TO RELATED APPLICATIONS

This application claims the benefit of U.S. Provisional Patent Application No. 60/821,840 filed on Aug. 9, 2006, which is incorporated by reference herein.

### FIELD OF THE INVENTION

The invention relates generally to respirators, ventilators and oscillators used to deliver inspiratory gas to a patient. The term “respirator” is used herein to refer to respirators, ventilators and oscillators collectively.

### BACKGROUND

Rebreathing circuits, such as so called “circle circuits”, are used in operating rooms to conserve volatile anesthetics. There has been a move in the anesthesia field to devices that have low fresh gas flow (“LFGF”) as a cost saving measure designed to reduce the amount of anesthesia used. While beneficial from an efficiency standpoint, some LFGF devices are tedious for the clinician to use because they require frequent attention to the fresh gas flow and manual adjustments of the fresh gas flow to achieve a desired amount of gas in the circuit.

Some newer anesthesia machines facilitate very low fresh gas flows, and provide nearly closed circuit anesthesia. Examples of these LFGF anesthesia machines are the Physioflex machine offered by Physio, Inc. and the machine described in U.S. Pat. No. 5,094,235. With these LFGF anesthesia machines, the clinician sets the desired oxygen concentration and either the desired inspired or expired anesthetic agent concentration. These LFGF anesthesia machines are not designed to allow precise control of the patient's ventilation or to facilitate spontaneous breathing, which might be needed in the Intensive Care Unit (“ICU”) setting, since they are not intended to work with the ventilators in common use in the ICU.

U.S. Pat. No. 6,675,799 (the “'799 Patent”) describes an isolation device having a movable partition and a housing disposed about the movable partition. The isolation device has a movable partition that divides the housing into a patient side and a respirator side. This isolation rebreather uses a partition biaser to help restore the partition that separates patient and ventilator sides of the device toward an equilibrium position. In doing so, the biaser may create a pressure difference between the patient side and the respirator side. This pressure difference may be used to control the flow of bias gas. However, that pressure difference may also distort the pressures applied by the ventilator to the patient, thereby making the device of the '799 Patent unsuitable for all situations.

### BRIEF DESCRIPTION OF THE DRAWINGS

The nature and objects of the invention will be made clearer with reference to the following detailed description taken in conjunction with the accompanying drawings, in which:

FIG. 1 is a schematic of a device according to the invention;

FIG. 2 is a top view of an isolation device according to the invention;

FIG. 3 is a cross-sectional view of the isolation device shown in FIG. 2 taken along the line 3-3;

FIG. 4 is a schematic drawing of an isolation device having a controller according to the invention;

FIGS. 5A-1, 5A-2 and 5A-3 are each a cross sectional view of an isolation device similar to that shown in FIG. 3, but having a partition biaser;

FIG. 5B is a cross sectional view similar to that shown in FIG. 5A-1, but having a different type of partition biaser; and

FIG. 6 is a flow chart of a method according to the invention.

### DETAILED DESCRIPTION OF THE INVENTION

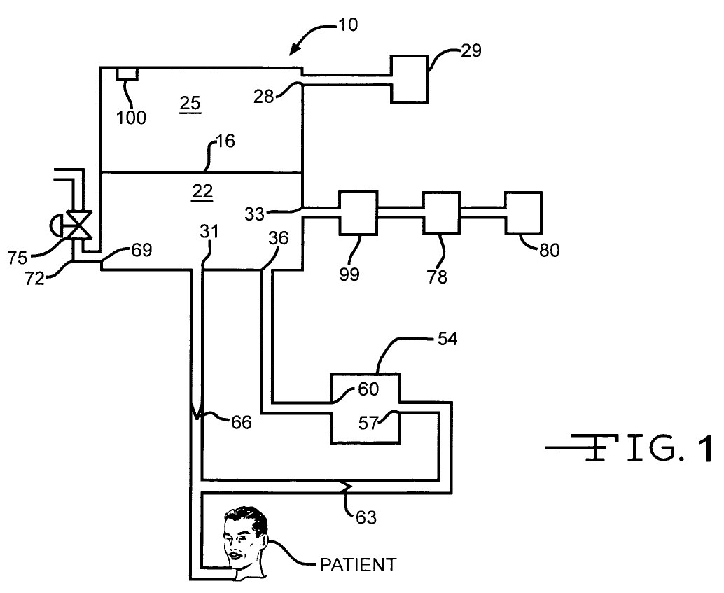

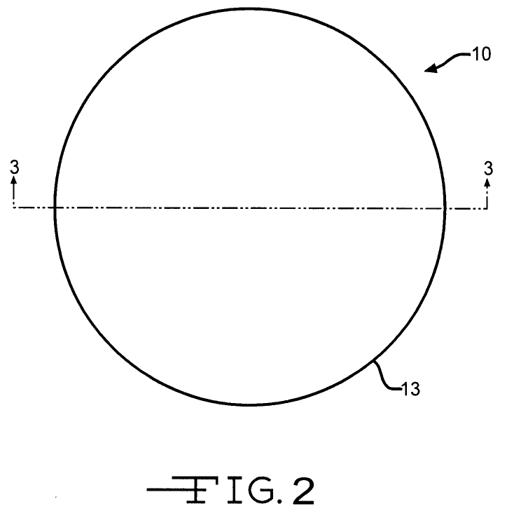

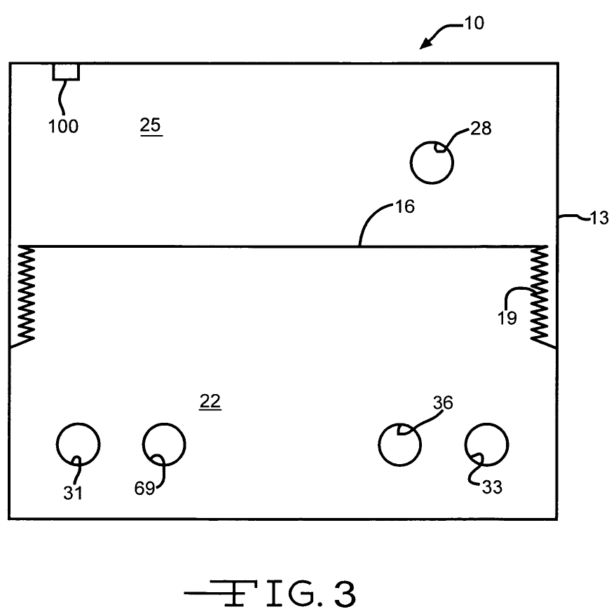

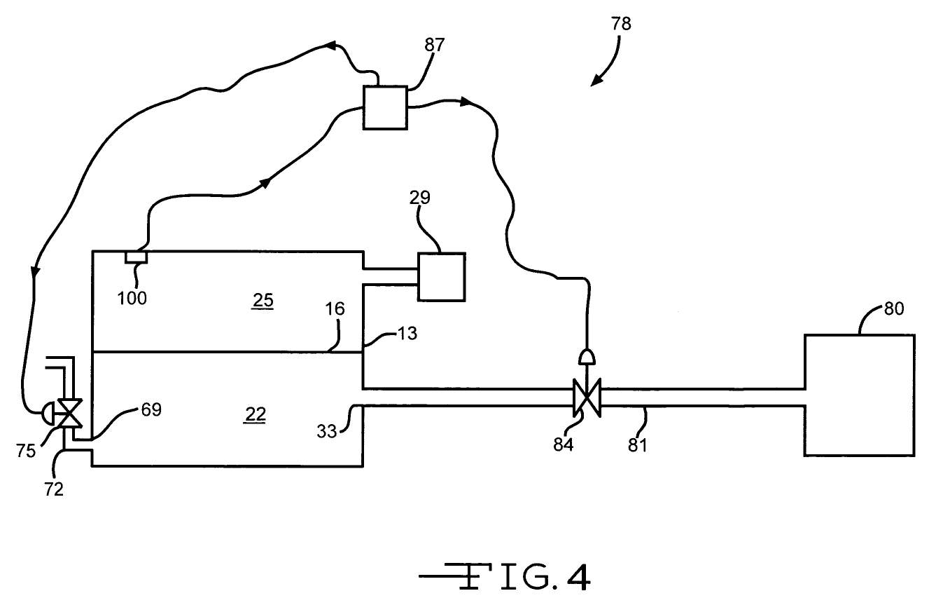

FIGS. 1, 2, and 3 illustrate aspects of an isolation device 10 according to the present invention. The isolation device 10 may have a housing 13 disposed about a movable partition 16. The partition 16 may include an accordion sleeve 19 joined to the housing 13 to allow movement of the partition 16. The partition 16 may be joined to the housing 13 to separate a patient side 22 of the housing 13 from a respirator side 25 of the housing. The housing 13 also may have a respirator orifice 28 on the respirator side 25 that is adaptable to be in pneumatic communication with a respirator 29. The housing 13 may also have a patient inspiration orifice 31 on the patient side 22 that is adaptable to be in pneumatic communication with a patient. The housing 13 may have a bias inflow orifice 33 on the patient side 22 that is adaptable to be in pneumatic communication with a source 80 of inspiratory gas, and an expiration return orifice 36 on the patient side 22. The housing 13 may be made of more than one piece, for example, the patient side 22 may be one piece and the respirator side 25 may be another piece.

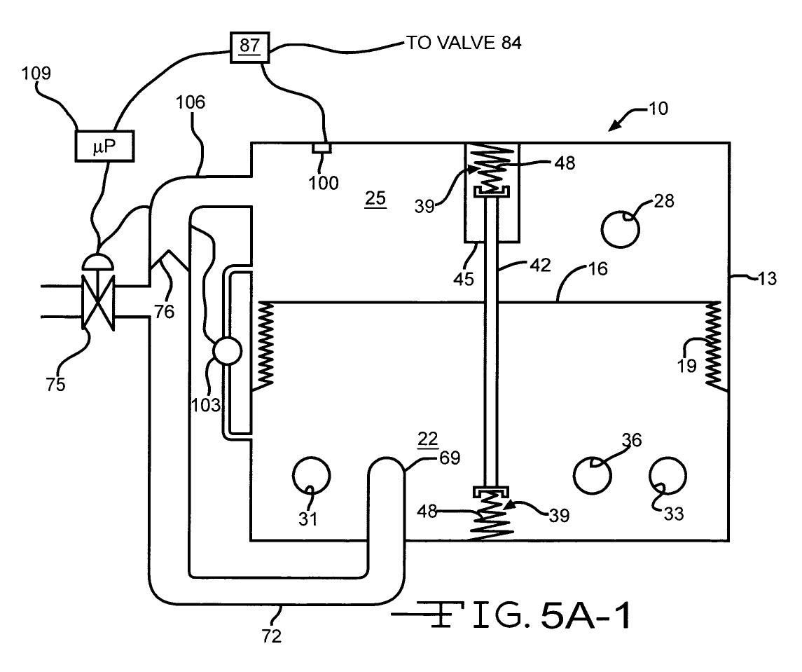

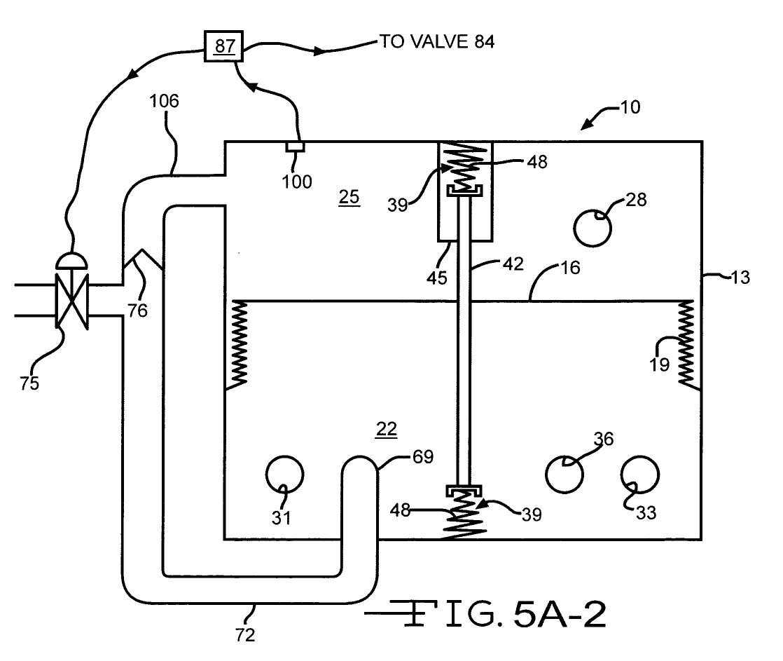

A controller 78 may be provided that is operable to regulate a flow of gas from an inspiratory gas source 80 to the bias inflow orifice 33. The controller 78 may be operable to achieve a desired flow rate for the flow of gas to the bias inflow orifice 33. FIG. 4 illustrates aspects of a controller according to the invention. The controller 78 may include a bias flow line 81 in pneumatic communication with the bias inflow orifice 33, and a bias flow control valve 84 in the bias flow line 81. The controller 78 may regulate the flow of gas to the bias inflow orifice 33 based on a position of the partition 16. The controller 78 may include a position transducer 87 operable to provide a signal corresponding to a position of the partition 16, and wherein the bias flow control valve 84 is positionable according to the signal.

A position sensor 100 may be used to detect the position of the partition 16. As an example, the position sensor 100 may be an ultrasound transducer, which is capable of indicating to the position transducer 87 the position of the partition 16. As another example, the partition 16 may include a contact and the housing 13 may have a high-position contact and a low-position contact, and then when the partition contact touches the high-position contact, the position transducer 87 may be signaled that the partition 16 is at the high position, and when the partition contact touches the low-position contact, the position transducer 87 may be signaled that the partition 16 is at the low position. Other types of position sensors 100 are well known, and could easily be employed as a position sensor 100.

In an embodiment of an isolation device 10 according to the invention, the housing 13 may have a bias release orifice 69 on the patient side 22, a release line 72 joined to the bias release orifice 69 and a release valve 75. The release valve 75 may be operable to allow gas to flow from the bias release orifice 69 to the atmosphere via the release line 72, for example by opening a gate in the release valve 75. The release valve 75 may be operable by the controller 78 to allow gas to flow from the bias release orifice 69 to the atmosphere when the partition 16 is too far to the respirator side 25. Furthermore, the release valve 75 may be operable to allow gas to flow from the bias release orifice 69 to the atmosphere during an expiration period.

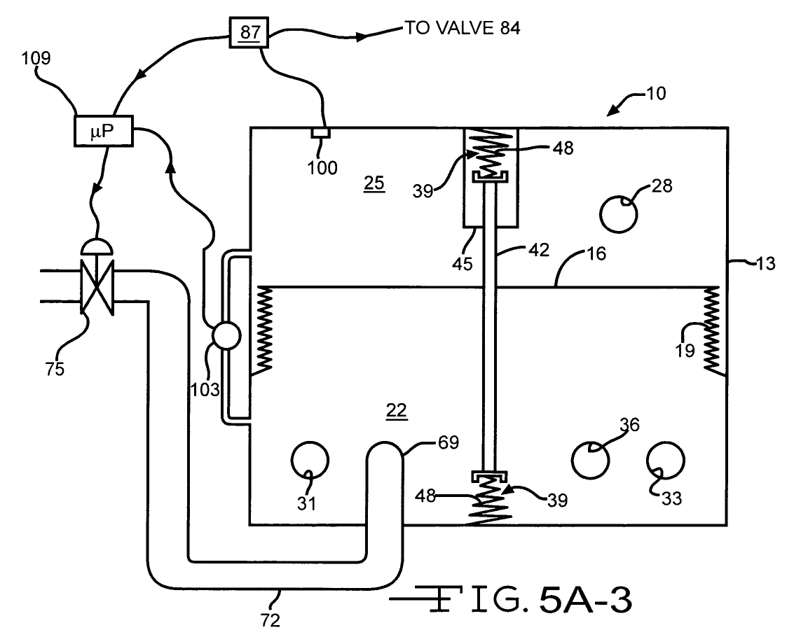

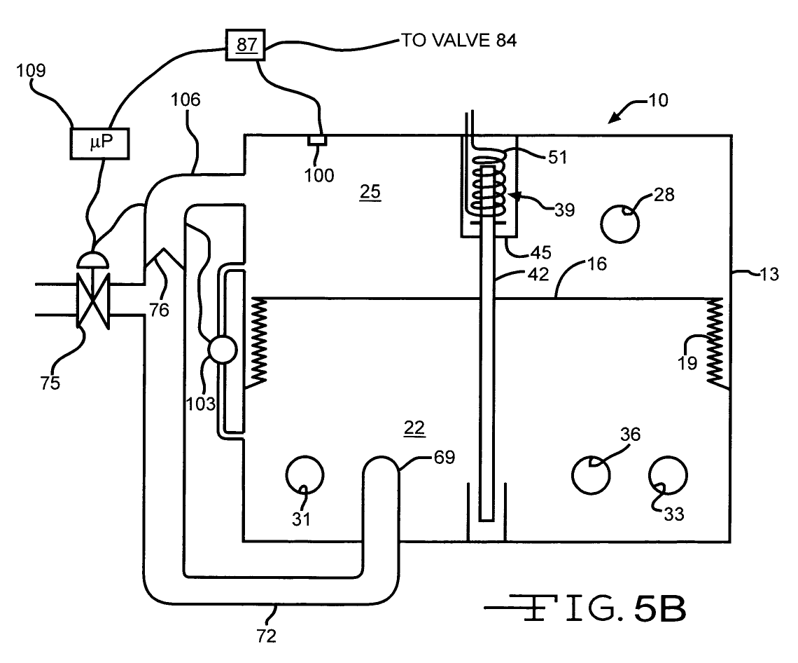

FIGS. 5A-1, 5A-2, 5A-3 and 5B illustrate embodiments of the invention which include a partition biaser 39 joined to the partition 16. The partition biaser 39 may be operable to bias the partition 16 to an undisplaced position during an expiration period. One such partition biaser 39 may have a movable rod 42 joined to the partition 16, and a spring 48 joined to the rod 42 to provide a force that biases the partition 16 to the undisplaced position. FIGS. 5A-1, 5A-2 and 5A-3 show such a partition biaser 39. FIG. 5B shows an embodiment with a different type of partition biaser 39, which has a solenoid 51 that may be used to provide the bias force. The solenoid 51 may be magnetically coupled to the rod 42. An abutment 45 may be provided to limit the travel of the rod 42, and therefore the partition 16.

FIGS. 5A-1, 5A-2 and 5B show embodiments in which the release line 72 includes a conduit 106 joining the patient side 22 with the respirator side 25. The conduit 106 may allow the patient side 22 to be at a pressure that is not significantly above the pressure on the respirator side 25. For example, when the partition biaser 39 moves the partition 16 toward the patient side 22, gas may be allowed to flow via the conduit 106 to the respirator side 25. In this manner, the pressure on the patient side 22 will not change much as a result of the partition biaser 39 moving the partition 16. A check valve 76 may be included to prevent gas flow from the respirator side 25 via the conduit 106.

The release valve 75 may be operable to open when the pressure on the patient side 22 is higher than the pressure on the respirator side 25. FIGS. 5A-1, 5A-3 and 5B show embodiments of such a device. One means of making this occur would use a pressure transducer 103 that is in communication with the release valve 75 for purposes of signaling to the release valve 75 when the release valve 75 should be open in order to reduce the pressure on the patient side 22. This operability may be in addition to that described above for controlling the position of the partition 16. As such, it may be beneficial for the signal from the position sensor 100 and the signal from the pressure transducer 103 to be sent to a microprocessor 109, which would then determine whether the release valve 75 should be open or closed. FIGS. 5A-1, 5A-3 and 5B show embodiments having a microprocessor 109. It should be noted that the embodiments depicted in 5A-2 and 5A-3 may use the partition biaser 39 shown in FIG. 5B—for brevity, figures corresponding to such embodiments have not been included in this application.

An isolation device 10 according to the invention may have a CO₂ scrubber 54 having an inlet 57 in pneumatic communication with the patient and an outlet 60 in pneumatic communication with the expiration return orifice 36. A check valve 63 may be provided in pneumatic communication with the scrubber 54 to prevent gas from traveling from the scrubber 54 toward the patient and to permit exhaled gas from the patient to flow through the scrubber 54. A check valve 66 may be provided in pneumatic communication with the patient inspiration orifice 31 to encourage exhaled gas from the patient to flow through the scrubber 54 and to permit gas from the patient inspiration orifice 31 to flow to the patient.

The bias inflow orifice 33 may be used to supply inspiratory gas from the inspiratory gas source 80 to the patient side 22 of the housing 13. A vaporizer, blender, mixer and/or nebulizer (shown as 99 in FIG. 1) may be placed in pneumatic communication with the bias inflow orifice 33, and these may be used to provide a therapeutic agent in the inspiratory gas.

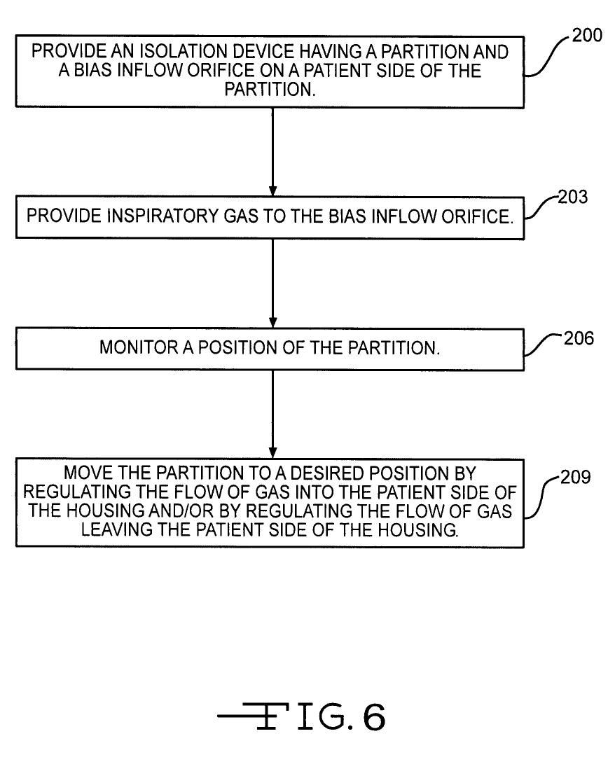

FIG. 6 shows a method according to the invention. A method according to the invention may include providing 200 an isolation device having a partition, and having a bias inflow orifice on a patient side of the partition. Inspiratory gas may be provided 203 to the bias inflow orifice, and a position of the partition may be monitored 206. When the partition is not in a desired position, the partition may be moved 209 to a desired position by increasing or decreasing the flow of inspiratory gas into the patient side of the housing and/or by increasing or decreasing the flow of gas leaving the patient side of the housing. The inspiratory gas may include a therapeutic agent.

The partition may be used to deliver the inspiratory gas to a patient. To do so, the partition may be moved in order to cause the inspiratory gas to leave the patient side via a patient inspiration orifice. Moving the partition in order to deliver gas to a patient may be accomplished by increasing a pressure in the respirator side. A respirator may be provided in pneumatic communication with a respirator orifice of the housing, and the respirator may be used to increase the pressure in the respirator side during inspiration.

The partition may also be moved, for example, by a partition biaser. In one method according to the invention, the partition is moved by the partition biaser prior to increasing the pressure with the respirator, so that the partition seeks an undisplaced position during expiration. This may cause gas to move from the patient side to the respirator side via a release line, or it may create a pressure difference between the respirator side and the patient side that may result in an increase in bias inflow to the patient side.

A method according to the invention may include moving the partition to allow expired gas from the patient to flow through the scrubber toward the expiration return orifice. This may be accomplished by decreasing a pressure on the respirator side. A respirator may be provided in pneumatic communication with the respirator orifice to decrease the pressure in the respirator side during expiration.

A method according to the invention may include providing a release line joined to the bias release orifice, and a release valve operable to allow gas to flow from the bias release orifice to the atmosphere via the release line, and the method may further include opening the release valve to move the partition toward the patient side.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What is claimed is:

1. An isolation device, comprising:
a movable partition;
a housing disposed about the movable partition, the housing having a respirator side on a first side of the partition, and having a patient side on a second side of the partition, and having (a) a respirator orifice on the respirator side, adaptable to be in pneumatic communication with a respirator, (b) a patient inspiration orifice on the patient side, adaptable to be in pneumatic communication with a patient, (c) a bias inflow orifice on the patient side, adaptable to be in pneumatic communication with a source of inspiratory gas, and (d) an expiration return orifice on the patient side;
a partition position sensor;
a CO₂ scrubber having an inlet in pneumatic communication with the patient and an outlet in pneumatic communication with the expiration return orifice; and
a controller operable to regulate a flow of gas to the bias inflow orifice based on a position of the partition.

2. The isolation device of claim 1, further comprising a partition biaser joined to the partition.

3. The isolation device of claim 2, wherein the partition biaser is operable to bias the partition to an undisplaced position.

4. The isolation device of claim 2, wherein the partition biaser includes a movable rod joined to the partition.

5. The isolation device of claim 4, further comprising an abutment limiting movement of the rod.

6. The isolation device of claim 2, wherein the housing further comprises a bias release orifice on the patient side, and the isolation device further comprises a release line joined to the bias release orifice, the release line including a conduit joining the patient side with the respirator side so that when a partition biaser moves the partition toward the patient side, gas is allowed to flow to the respirator side.

7. The isolation device of claim 1, further comprising a check valve in pneumatic communication with the scrubber to prevent gas from traveling from the scrubber toward the patient and to permit exhaled gas from the patient to flow through the scrubber.

8. The isolation device of claim 1, further comprising a check valve in pneumatic communication with the patient inspiration orifice to encourage exhaled gas from the patient to flow through the scrubber and to permit gas from the patient inspiration orifice to flow to the patient.

9. The isolation device of claim 1, wherein the housing further comprises a bias release orifice on the patient side, and the isolation device further comprises:
a release line joined to the bias release orifice; and
a release valve operable to allow gas to flow from the bias release orifice to atmosphere.

10. The isolation device of claim 9, wherein the release valve is operable to allow gas to flow from the bias release orifice to atmosphere during an expiration period.

11. The isolation device of claim 9, wherein the release valve is operable to release gas to atmosphere based on a position of the partition.

12. The isolation device of claim 11, wherein the release valve is operable to release gas to atmosphere based on the relative pressures between the first side of the partition and the second side of the partition.

13. The isolation device of claim 1, wherein the controller is operable to achieve a desired flow rate for the flow of gas to the bias inflow orifice.

14. The isolation device of claim 1, wherein the controller includes a bias flow line in pneumatic communication with the bias inflow orifice, and a bias flow control valve in the bias flow line.

15. The isolation device of claim 1, wherein the bias inflow orifice is also in pneumatic communication with a vaporizer.

16. The isolation device of claim 1, wherein the bias inflow orifice is also in pneumatic communication with a blender.

17. The isolation device of claim 1, wherein the bias inflow orifice is also in pneumatic communication with a mixer.

18. The isolation device of claim 1, wherein the partition includes an accordion sleeve joined to the housing.

19. An isolation device, comprising:
a movable partition;
a housing disposed about the movable partition, the housing having a respirator side on a first side of the partition, and having a patient side on a second side of the partition, and having (a) a respirator orifice on the respirator side, adaptable to be in pneumatic communication with a respirator, (b) a patient inspiration orifice on the patient side, adaptable to be in pneumatic communication with a patient, (c) a bias inflow orifice on the patient side, adaptable to be in pneumatic communication with a source of inspiratory gas, (d) an expiration return orifice on the patient side, and (e) a bias release orifice on the patient side;
a partition position sensor;
a CO₂ scrubber having an inlet in pneumatic communication with the patient and an outlet in pneumatic communication with the expiration return orifice; and
a controller operable to regulate a flow of gas from the bias release orifice based on a position of the partition.

20. A method of controlling a partition, comprising:
providing an isolation device having (a) a movable partition, (b) a housing disposed about the movable partition, the housing having (i) a respirator side on a first side of the partition (ii) a patient side on a second side of the partition, (iii) a respirator orifice on the respirator side, adaptable to be in pneumatic communication with a respirator, (iv) a patient inspiration orifice on the patient side, adaptable to be in pneumatic communication with a patient, (v) a bias inflow orifice on the patient side, adaptable to be in pneumatic communication with a source of inspiratory gas, (vi) an expiration return orifice on the patient side, and (vii) a bias release orifice on the patient side, and (c) a CO₂ scrubber having an inlet in pneumatic communication with the patient and an outlet in pneumatic communication with the expiration return orifice;
providing an inspiratory gas to the bias inflow orifice;
monitoring a position of the partition; and
moving the partition to a desired position by regulating the flow of inspiratory gas to the patient side and/or from the patient side.
